(19) United States
(12) Patent Application Publication
(10) Pub. No.: US 2026/0245701 A1
(43) Pub. Date: Aug. 20, 2026

(54) DYNAMIC TASK GENERATION IN VIRTUAL ENVIRONMENT TO ASSESS SYMPTOMS OF COGNITIVE DISORDERS

(71) Applicant: International Business Machines Corporation, Armonk, NY (US)

(72) Inventors: Guang Han SUI, Beijing (CN); Peng Hui JIANG, Beijing (CN); Su LIU, Austin, TX (US); Jun SU, Beijing (CN); Yu ZHU, Beijing (CN); Jun Feng DUAN, Beijing (CN)

(21) Appl. No.: 19/056,327

(22) Filed: Feb. 18, 2025

(51) Int. Cl. G16H 20/70; G06F 3/01; G06T 13/40; G16H 40/67

(52) U.S. Cl. CPC G16H 20/70; G06F 3/011; G06T 13/40; G16H 40/67

(57) ABSTRACT

A method, and associated system and computer program product, is disclosed that includes prompting a user to perform a first task within a virtual environment. The first task is suggestive of whether the user exhibits one or more symptoms of a set of predetermined symptoms indicative of a cognitive disorder. The method further includes assessing performance of the first task responsive to user inputs received from one or more input devices, and determining, based on the performance of the first task, whether performance of one or more additional tasks is needed to conclusively assess whether the user exhibits any of the one or more symptoms. The method further includes reporting, based on the performance of the first task and the performance of the one or more additional tasks when needed, an assessment of whether the user exhibits any of the one or more symptoms.

### BACKGROUND

The present disclosure relates to virtual environments, and more specifically, to generating tasks within virtual environments to assess whether a user exhibits symptoms of cognitive disorders such as Alzheimer's disease.

Alzheimer's disease is a disorder of the human brain, characterized by the presence of abnormal protein deposits (“plaques” and “tangles”) on nerve cells. These protein deposits damage the nerve cells and eventually cause the nerve cells to die. The hippocampus, which is vital for forming memories, is typically one of the first areas of the brain to be affected. Thus, as Alzheimer's disease progresses, the disease gradually destroys the memory and cognitive ability of the patient, and degrades the patient's ability to perform everyday tasks.

While no cure is known for Alzheimer's disease, medications and other interventions have been shown to be effective in slowing the progression of symptoms and preserving daily functioning of the patient. Thus, it can be beneficial to diagnose Alzheimer's disease and begin treatment as early as possible. However, delays in beginning treatment may occur as the progression of symptoms may be imperceptible to, or disregarded by, the patient or others.

### SUMMARY

According to one embodiment of the present disclosure, a method includes prompting a user to perform a first task within a virtual environment. The first task is suggestive of whether the user exhibits one or more symptoms of a set of predetermined symptoms indicative of a cognitive disorder. The method further includes assessing performance of the first task responsive to user inputs received from one or more input devices, and determining, based on the performance of the first task, whether performance of one or more additional tasks is needed to conclusively assess whether the user exhibits any of the one or more symptoms. The method further includes reporting, based on the performance of the first task and the performance of the one or more additional tasks when needed, an assessment of whether the user exhibits any of the one or more symptoms.

According to one embodiment of the present disclosure, a system includes one or more computer processors configured to prompt a user to perform a first task within a virtual environment. The first task is suggestive of whether the user exhibits one or more symptoms of a set of predetermined symptoms indicative of a cognitive disorder. The one or more computer processors are further configured to assess performance of the first task responsive to user inputs received from one or more input devices, and determine, based on the performance of the first task, whether performance of one or more additional tasks is needed to conclusively assess whether the user exhibits any of the one or more symptoms. The one or more computer processors are further configured to report, based on the performance of the first task and the performance of the one or more additional tasks when needed, an assessment of whether the user exhibits any of the one or more symptoms.

According to one embodiment of the present disclosure, a computer program product includes one or more computer-readable storage media, and program instructions stored on the one or more computer-readable storage media to perform operations that include prompting a user to perform a first task within a virtual environment. The first task is suggestive of whether the user exhibits one or more symptoms of a set of predetermined symptoms indicative of a cognitive disorder. The operations further include assessing performance of the first task responsive to user inputs received from one or more input devices, and determining, based on the performance of the first task, whether performance of one or more additional tasks is needed to conclusively assess whether the user exhibits any of the one or more symptoms. The operations further include reporting, based on the performance of the first task and the performance of the one or more additional tasks when needed, an assessment of whether the user exhibits any of the one or more symptoms.

### BRIEF DESCRIPTION OF THE DRAWINGS

FIG. 1 is a block diagram of an example computer environment for use in conjunction with one or more disclosed embodiments for assessing symptoms of a cognitive disorder within a virtual environment, according to one or more embodiments.

FIG. 2 is a block diagram of an example system for assessing symptoms of a cognitive disorder within a virtual environment, according to one or more embodiments.

FIG. 3 is an example method of assessing symptoms of a cognitive disorder within a virtual environment, according to one or more embodiments.

FIG. 4A is a diagram illustrating a map view of an example virtual environment, according to one or more embodiments.

FIG. 4B is a diagram illustrating an avatar approaching a first intersection within the virtual environment, according to one or more embodiments.

FIG. 4C is a diagram illustrating an avatar approaching a second intersection within the virtual environment, according to one or more embodiments.

FIG. 4D is a diagram illustrating an avatar approaching a first intersection within the virtual environment, according to one or more embodiments.

### DETAILED DESCRIPTION

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the aspects, features, embodiments and advantages disclosed herein are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.”

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

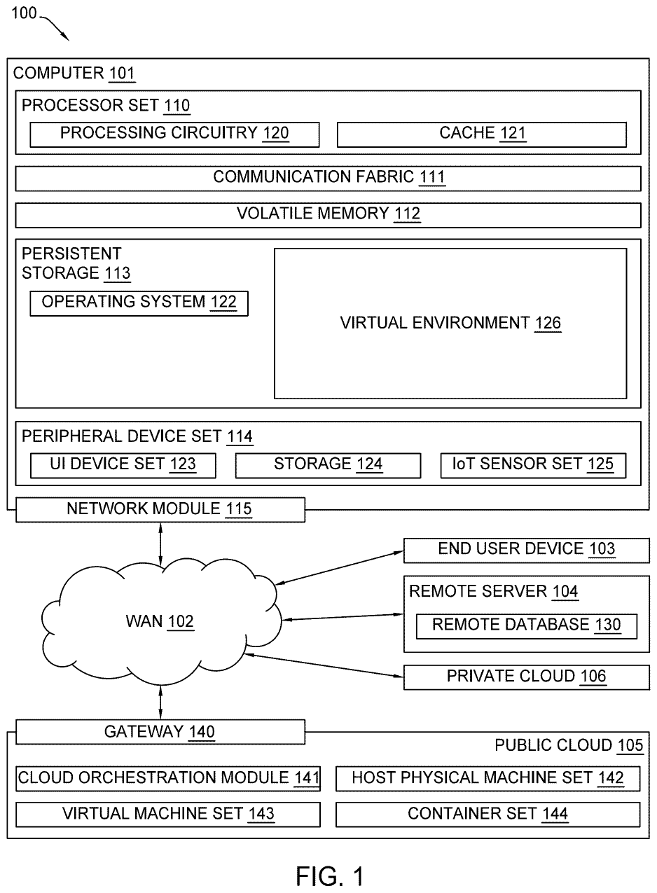

Referring now to FIG. 1, computing environment 100 contains an example of an environment for the execution of at least some of the computer code involved in performing the inventive methods, such as improved virtual environment code 126 that generates and presents tasks within the virtual environment for assessing whether a user exhibits any symptoms of a cognitive disorder, such as Alzheimer's disease. In addition to the block representing the virtual environment code 126, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and the block representing the virtual environment code 126, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in the block representing the virtual environment code 126 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in the block representing the virtual environment code 126 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

CLOUD COMPUTING SERVICES AND/OR MICROSERVICES (not separately shown in FIG. 1): private and public clouds 105, 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (Saas) where software is centrally hosted and allocated on a subscription basis. SaaS is also known as on-demand software, web-based software, or web-hosted software. Four technological sub-fields involved in cloud services are: deployment, integration, on demand, and virtual private networks.

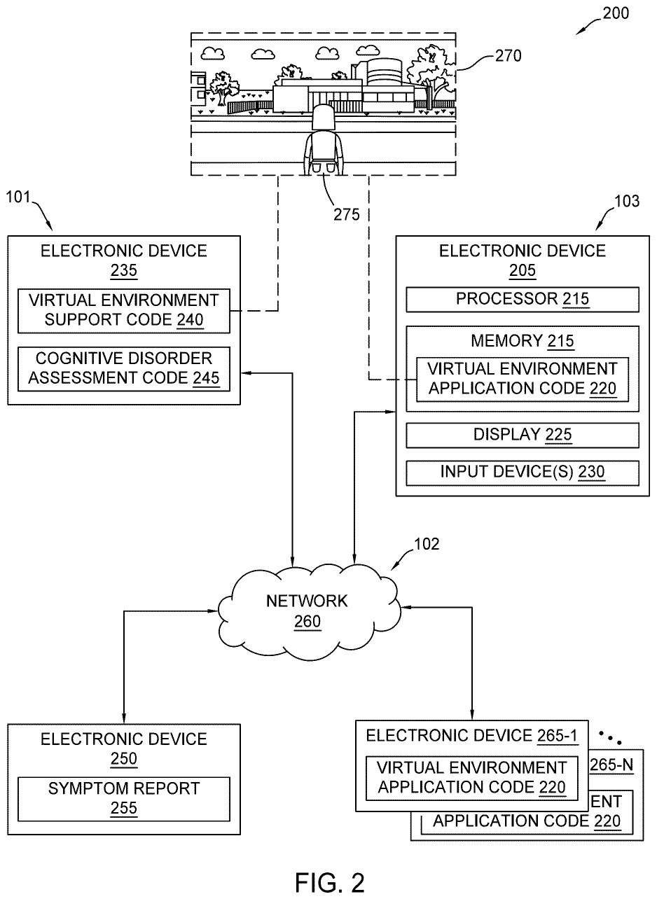

FIG. 2 is a block diagram of an example system 200 for assessing symptoms of a cognitive disorderwithin a virtual environment, according to one or more embodiments. The features illustrated in FIG. 2 may be used in conjunction with other embodiments, e.g., representing one example implementation of the computing environment 100 of FIG. 1.

The system 200 includes an electronic device 205 (also referred to as “user device 205”) representing an example of the end user device 103, an electronic device 235 (also referred to as “provider device 235”) representing an example of the computer 101, and an electronic device 250 (also referred to as “reporting device 250”). The electronic devices 205, 235, 250 are communicatively connected by a network 260 representing an example of the WAN 102 and may encompass one or more WANs and/or one or more LANs.

As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

The electronic device comprises one or more processors and a memory. The one or more processors are any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors are not limited to a single processing device and may encompass multiple processing devices.

The one or more processors may include other hardware that operates software to control and process information. In some aspects, the one or more processors execute software stored in the memory to perform any of the functions described herein. The one or more processors control the operation and administration of the electronic device by processing information (e.g., information received from input devices and/or communicatively coupled electronic devices).

The memory may store, either permanently or temporarily, data, operational software, or other information for the one or more processors. The memory may include any one or a combination of volatile or non-volatile local or remote devices suitable for storing information. For example, the memory may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD, or a flash drive. In particular embodiments, the software may include an application executable by the one or more processors to perform one or more of the functions described herein.

As shown, the electronic device 205 comprises one or more computer processors 210 and a memory 215 that stores virtual environment application code 220. The electronic device 205 further comprises a display 225 and one or more input devices 230, which may be integrated with or implemented separately from the electronic device 205. In one non-limiting example, the electronic device 205 is implemented as a desktop computer having a separate monitor as the display 225 and having external input devices such as a mouse and keyboard. In another non-limiting example, the electronic device 205 is implemented as a smartphone having an integrated display 225 and an integrated touchscreen as the input device 230. In yet another non-limiting example, the electronic device 205 is implemented as, or is connected to, a wearable device such as an augmented reality (AR) or virtual reality (VR) headset that displays and/or receives inputs from the wearer.

The one or more computer processors 210 execute the virtual environment application code 220 to generate a virtual environment 270 with which the user interacts using the display 225 and the one or more input devices 230. In some embodiments, the user interacts with the virtual environment 270 via an avatar 275, which is an electronic image that represents the user and that can be controlled by the user to traverse the virtual environment 270. In other embodiments, the user may view the virtual environment 270 with a first-person perspective, such that the avatar 275 is not normally observed by the user.

In some cases, similar virtual environment application code 220 may be executed by one or more additional electronic devices 265-1, . . . , 265-N connected to the network 260 to enable one or more additional users to interact with the same virtual environment 270 as the user of the electronic device 205. The one or more additional users may have respective avatars 275 that have distinctive appearances and that may interact with each other within the virtual environment 270. For example, when two avatars 275 encounter each other within the virtual environment 270, the avatars 275 may interact with each other using a predetermined set of inputs and/or may enable communication between the users of the respective avatars 275 (e.g., through voice and/or text chat).

In some embodiments, the virtual environment application code 220 of the user device 205 generates and maintains the virtual environment 270 independently of other electronic devices. In other embodiments, and as shown, the virtual environment application code 220 generates and maintains the virtual environment 270 in conjunction with virtual environment support code 240 that executes on the provider device 235 and/or the virtual environment application code 220 executed by the one or more additional electronic devices 265-1, . . . , 265-N. The virtual environment support code 240 may provide any aspects of the virtual environment 270 and/or may extend the functionality available within the virtual environment 270, such as providing the ability to conduct purchases and/or other monetary transactions over the network 260.

In some embodiments, the virtual environment 270 defines a number of different virtual spaces to which the user can navigate their avatar 275 to interact with different functionality of the virtual environment 270. For example, the different virtual spaces may be implemented as different rooms or buildings that provide different games, puzzles, activities, and so forth that allow the user to tailor their experience within the virtual environment 270. In some embodiments, the virtual environment 270 may further define a common space that is navigable by the avatar 275 to access the different virtual spaces, and may be implemented in any suitable form such as a road, walkway, lobby, waiting room, and so forth.

In some embodiments, the provider device 235 comprises cognitive disorder assessment code 245 that prompts users to perform specific tasks within the virtual environment 270, where the tasks are suggestive (or probative) of whether the user exhibits one or more symptoms of a set of predetermined symptoms indicative of a cognitive disorder. The tasks may be predefined and stored by the cognitive disorder assessment code 245, or may be dynamically generated by the cognitive disorder assessment code 245, e.g., to specifically assess certain symptoms or warning signs, which are discussed in greater detail below. In some embodiments, the tasks may be selected or generated based on historical interaction information for the user within the virtual environment 270. For example, the cognitive disorder assessment code 245 may generate tasks that have some similarity to previous interactions of the user, or that take place in a familiar setting within the virtual environment 270.

The tasks generated by the cognitive disorder assessment code 245 may use any functionality of the virtual environment 270. Some examples of the tasks include navigating the avatar 275 to a predefined location within the virtual environment 270, interacting with one or more other avatars or non-playable characters (NPCs) in the virtual environment 270, performing any of the functionality defined by the different virtual spaces, and so forth.

The cognitive disorder assessment code 245 further assesses the user's performance of the tasks responsive to user inputs received from the one or more input devices 230. Although described specifically using the example of Alzheimer's disease, the tasks generated and presented by the cognitive disorder assessment code 245 may be similarly advantageous to assess the presence (or absence) and/or progress of other types of cognitive disorders. The cognitive disorder assessment code 245 may perform the assessment algorithmically or through applying the user inputs to a model (e.g., trained using previous task performances by healthy individuals, individuals exhibiting a diagnosed cognitive disorder, and so forth). The cognitive disorder assessment code 245 may present the tasks in any suitable form, such as tasks constrained to a particular virtual space of the virtual environment 270, tasks constrained to the common space, and so forth. Further, in some embodiments, the virtual environment support code 240 may require that the tasks presented by the cognitive disorder assessment code 245 be completed prior to enabling other aspects of the virtual environment 270.

The Centers for Disease Control (CDC) and the Alzheimer's Association provide a list of ten (10) warning signs (or risk factors) that suggest the presence of Alzheimer's disease: (1) memory loss that disrupts daily life, (2) challenges in planning or solving problems, (3) difficulty completing familiar tasks at home, at work, or at leisure, (4) confusion with time or place, (5) trouble understanding visual images and spatial relations, (6) new problems with words in speaking or writing, (7) displacing things and not being able to retrace steps, (8) decreased or poor judgment, (9) withdrawal from work or social activities, and (10) changes in mood or personality.

In some embodiments, the tasks generated by the cognitive disorder assessment code 245 are suggestive of whether the user exhibits one or more symptoms of a set of predetermined symptoms indicative of Alzheimer's disease. In some cases, the set of predetermined symptoms corresponds directly to the ten warning signs. However, it is contemplated that the cognitive disorder assessment code 245 includes a set of predetermined symptoms that overlap partially with the ten warning signs, as it may be difficult or impractical to generate tasks within the virtual environment 270 that are suitable to directly assess one or more of the warning signs (or aspects thereof). For example, it may be impractical to assess warning sign (10) (changes in mood or personality of the user) within the particular configuration of the virtual environment 270.

In some embodiments, the set of predetermined symptoms indicative of Alzheimer's disease comprises one or more of: forgetting recently presented information, decreased concentration, difficulty completing a repeated task, confusion about time or place, difficulty with vision, misplacing items, and inability to retrace a path. Individual symptoms within the set may generally correspond to one or more of the warning signs. For example, a decreased concentration symptom may have a substantial correlation to at least the warning signs (1), (2), (3), (4), (5), (6), and (7).

In some embodiments, each task that is generated by the cognitive disorder assessment code 245 has a respective plurality of correlation values r for some or all of the set of predetermined symptoms, such that assessing the user's performance of the task(s) contributes to an overall score for the symptom across the task(s). The correlation values may be defined in any suitable range, such as −1.0 to 1.0, 0 to 1.0, and so forth. In some embodiments, each symptom may further have a second plurality of correlation values that further relate the symptom to some or all of the ten warning signs, such that the cognitive disorder assessment code 245 may further calculate overall score(s) for some or all of the ten warning signs.

In one example, a task that includes navigating an avatar 275 to a predefined location within the virtual environment 270. The navigating task might correlate strongly (e.g., |r| between 0.6 and 0.8) to the following symptoms: forgetting recently presented information, decreased concentration, confusion about time or place, and difficulty with vision. If the same task is presented to the user more than once (whether in a same session or over multiple sessions), the task may also correlate strongly to difficulty completing a repeated task and inability to retrace a path. To calculate scores for the symptoms, the correlation values r may be multiplied by a value representing the user's performance of the task, such as a binary value (0 or 1, −1 or 1) for a successful or unsuccessful completion, a binary value for whether the task was completed within a time criterion, a continuous value that is based one or more metrics (e.g., a time to completion, how much visual guidance is presented with the task), and so forth. Generating the overall score for a particular symptom may be based on partial scores from the different tasks presented to the user, e.g., as a sum of the partial scores, an average of the sum, a largest partial score, and so forth.

Should the overall score for a particular symptom exceed a threshold, the cognitive disorder assessment code 245 may conclusively assess that the user exhibits the symptom. The threshold may be any suitable value, and may be positive or negative to confirm that the characterization of the conclusive assessment is affirmative (symptom exhibited) or negative (symptom not exhibited). For example, if the threshold value is ±0.6, values that are less than 0.6 and greater than −0.6 may be considered a non-conclusive assessment. If the overall score does not exceed the threshold, the cognitive disorder assessment code 245 may determine whether performance of one or more additional tasks is needed to make a conclusive assessment.

In some embodiments, the next task may be a repetition of the first task with the same or increased difficulty (such as removing some or all of the visual guidance), or another task that is selected or dynamically generated. For example, the next task may be selected from a plurality of candidate tasks, or may be dynamically generated, based on a stronger correlation of the candidate task with the symptom(s) whose overall score(s) did not meet the threshold for a conclusive assessment.

Performance of the one or more additional tasks may continue until one or more suitable criteria are met, such as exceeding the threshold for the symptom(s), reaching a maximum number of tasks, having less than a minimum amount of change in the overall score per iteration, and so forth.

Based on the performance of the first task and (optionally) the one or more additional tasks, the cognitive disorder assessment code 245 generates an symptom report 255 that is an assessment of whether the user exhibits any of the one or more symptoms. The assessment may be presented in any suitable form, such as reporting the values of the set of predetermined symptoms (or of the ten warning signs), reporting those symptoms having a conclusive assessment, reporting an overall characterization of the one or more symptoms, and so forth. The symptom report 255 may further include a recommendation as to whether the user should seek a formal medical assessment.

The cognitive disorder assessment code 245 may transmit the symptom report 255 to the user device 205 and/or to the reporting device 250 that in some cases corresponds to an entity other than the user (e.g., a colleague or family member, a medical provider). In some cases, the user may authorize the transmission of the symptom report 255 to the other entity (or entities) within the virtual environment application code 220, e.g., as one or more settings.

Beneficially, the operation of the cognitive disorder assessment code 245 by the provider device 235, which in some cases may be integrated with the operation of the virtual environment support code 240, allows for an inexpensive, regular assessment of the user that may be presented relatively seamlessly within the user's routine experience in the virtual environment 270. Presenting the assessment in this manner may be effective to reduce delays in the diagnosis or treatment of a cognitive disorder in the user, as the presence or progression of certain risk factors that might otherwise be imperceptible or disregarded can be recognized and tracked with greater consistency.

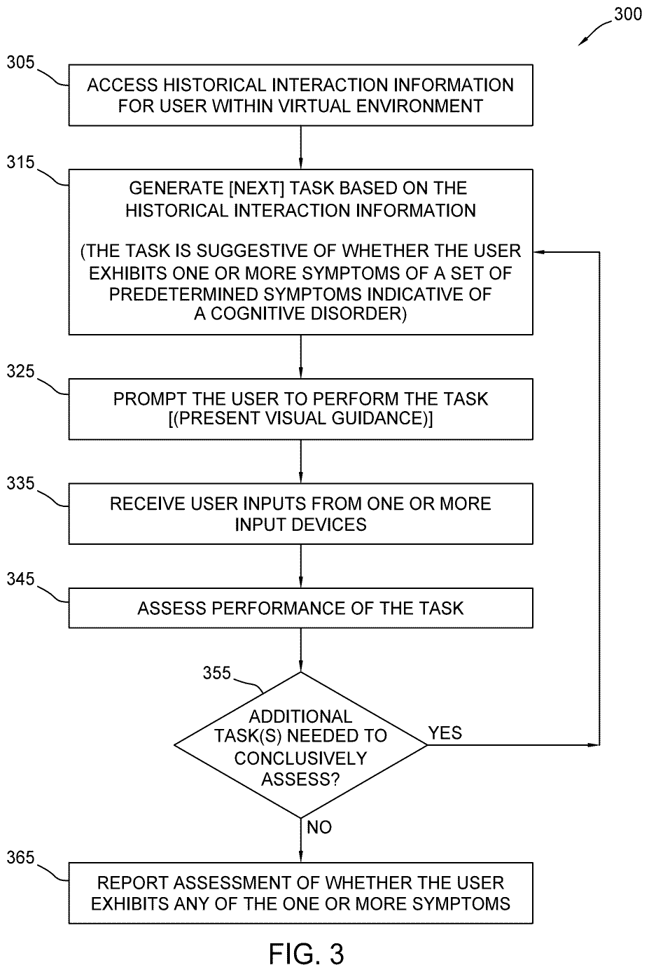

FIG. 3 is an example method 300 of assessing symptoms of a cognitive disorder within a virtual environment, according to one or more embodiments. The features of the method 300 may be used in conjunction with other embodiments, such as being performed by the virtual environment application code 220, the virtual environment support code 240, and/or the cognitive disorder assessment code 245.

The method 300 begins at block 305, where the cognitive disorder assessment code 245 accesses historical interaction information for the user within the virtual environment 270. At block 315, the cognitive disorder assessment code 245 generates a task based on the historical interaction information. In some embodiments, the cognitive disorder assessment code 245 generates the task to have some similarity to one or more previous interactions of the user, such as occurring near a same location in the virtual environment 270 as the one or more previous interactions. In some embodiments, generating the task comprises selecting the task from a plurality of (predefined) candidate tasks. In other embodiments, generating the task comprises dynamically generating the task.

The task is suggestive of whether the user exhibits one or more symptoms of a set of predetermined symptoms indicative of a cognitive disorder. In some embodiments, the task has a respective plurality of correlation values r for some or all of the set of predetermined symptoms. The set of predetermined symptoms may overlap with, or in some cases may correspond directly to, the ten warning signs that are discussed above. In some embodiments, a second plurality of correlation values relate each of the symptoms to the different warning signs.

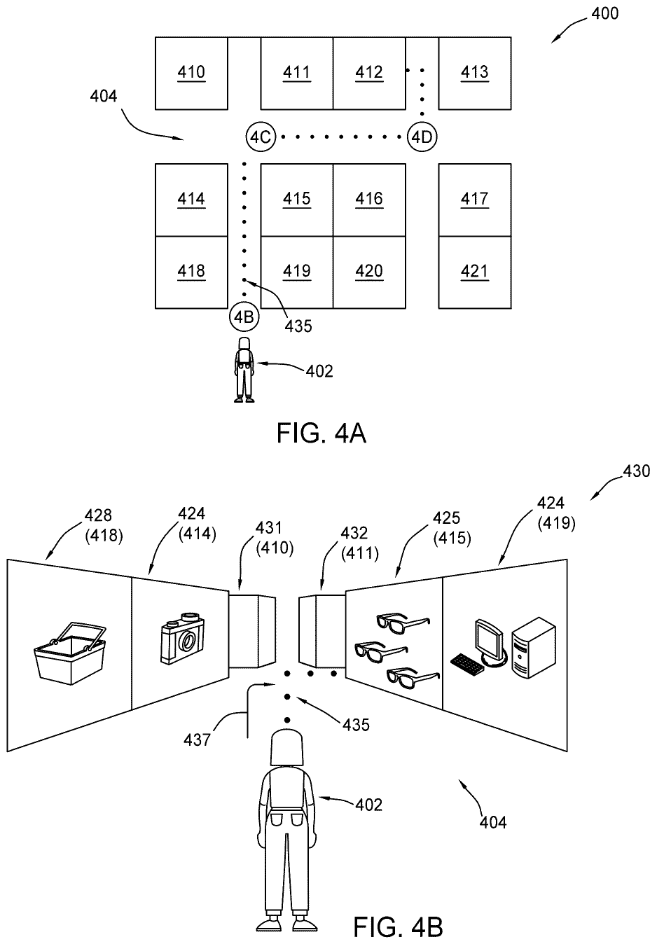

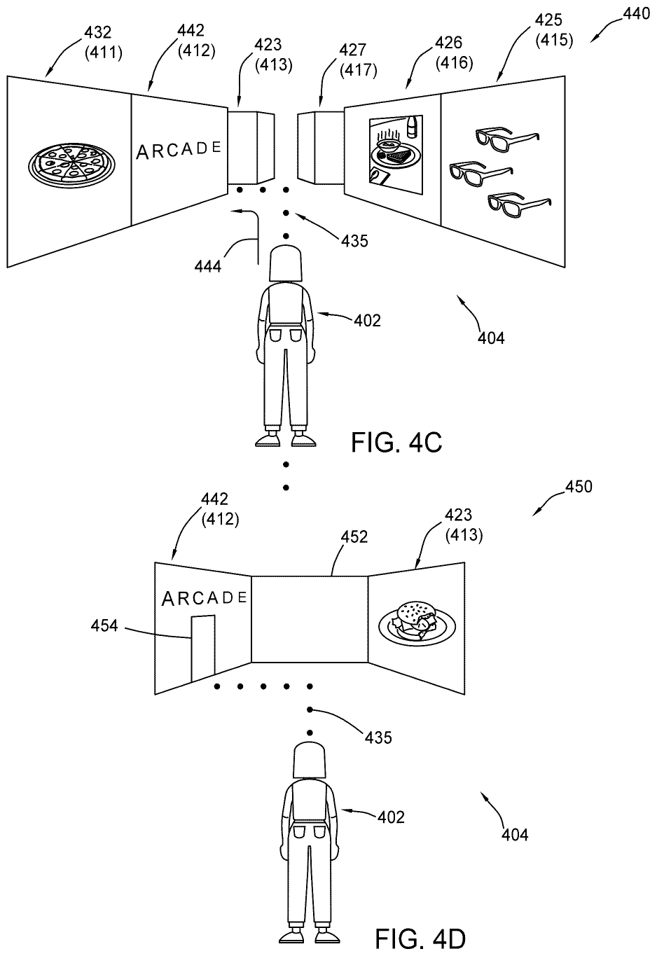

At block 325, the cognitive disorder assessment code 245 prompts the user to perform the task within the virtual environment 270. Refer also to FIG. 4A, which is a diagram 400 illustrating a map (overhead) view of an example virtual environment 270, according to one or more embodiments.

In the diagram 400, a plurality of rooms 410, 411, . . . , 420, 421 are arranged within the virtual environment 270 and are accessible by a corridor 404. Each of the rooms 410, 411, . . . , 420, 421 represents a virtual space that provides a respective functionality to the virtual environment 270, such as different games, puzzles, activities, stores, and so forth. An avatar 402 (one example of the avatar 275) represents the user within the virtual environment 270 and may be controlled by the user to navigate the corridor 404 to a desired room.

In some embodiments, the task includes navigating the avatar 402 to a predefined location within the virtual environment 270 (in this example, to the room 412). In some embodiments, the prompting the user to perform the first task includes presenting visual guidance to the predefined location. In diagram 400, the visual guidance comprises a path 435 that is depicted as a breadcrumb trail for the user.

FIGS. 4B, 4C, and 4D illustrate the avatar 402 as it encounters successive intersections of the corridor 404 that are along the path 435. In diagram 430 of FIG. 4B, different rooms 410, 411, 414, 415, 418, 419 are depicted with respective facades 431, 432, 424, 425, 428, 429 (depicted as different storefronts) that the avatar 402 may approach and may enter through to interact with the functionality of the rooms 410, 411, 414, 415, 418, 419. The avatar 402 approaches a first intersection at which the avatar 402 must make a right turn to remain on the path 435. In diagram 430, the visual guidance presented to the user comprises the path 435 as well as a first turn indicator 437 guiding the user to turn right.

At block 335, the virtual environment application code 220 receives user input(s) from the one or more input devices 230. For example, the user input(s) may control the avatar 402 to continue along the path 435 (e.g., to negotiate the right turn successfully).

In diagram 440 of FIG. 4C, the rooms 411, 412, 413, 415, 416, 417 are depicted with respective facades 432, 442, 423, 425, 426, 427. The avatar 402 approaches a second intersection at which the avatar 402 must make a left turn to remain on the path 435. In diagram 440, the visual guidance presented to the user comprises the path 435 as well as a second turn indicator 444 guiding the user to turn left.

In diagram 450 of FIG. 4D, the rooms 412, 413 are depicted with respective facades 442, 423. A boundary 452 extends between the facades 442, 423 and defines an extent of the corridor 404 (e.g., the avatar 402 is prevented from continuing travel beyond the facades 442, 423). In diagram 450, the visual guidance presented to the user comprises the path 435 as well as a visual indicator 454 that identifies the facade 442 and/or the room 412 as the predefined location to complete the task. The visual indicator 454 is depicted as an entryway to suggest to the user that the avatar 402 be directed into the room 412. However, other types of visual indicators 454 are also contemplated, such as a visual effect that distinguishes the facade 442 and/or the room 412 (e.g., highlight, outline) from other visual elements in the virtual environment 270.

At block 345, the cognitive disorder assessment code 245 assesses performance of the task responsive to the user inputs. In some embodiments, the cognitive disorder assessment code 245 assigns a binary value indicating a successful or unsuccessful completion of the task, whether the task was completed within a time criterion, and so forth. In other embodiments, the cognitive disorder assessment code 245 assigns a continuous value that is based one or more metrics (e.g., a time to completion, how much visual guidance is presented with the task) characterizing the completion or non-completion of the task. In some cases, the value(s) representing performance of the task is/are multiplied by the plurality of correlation values r to generate scores for some or all of the predetermined symptoms. Other techniques for assessing performance are also contemplated.

At block 355, the cognitive disorder assessment code 245 determines, based on the performance of the task, whether performance of one or more additional tasks is needed to conclusively assess whether the user exhibits any of the one or more symptoms. In some embodiments, determining whether performance of the one or more additional tasks is needed comprises comparing the generated scores with a threshold. If the score does not exceed the threshold, the cognitive disorder assessment code 245 may determine that one or more additional tasks are needed (“YES”) and flow proceeds to the block 315 where a next task is generated.

In some embodiments, the next task may be a repetition of the first task with the same or increased difficulty, such as removing some or all of the visual guidance. For example, returning to the example of FIGS. 4A-4D, removing some or all of the visual guidance may include altering or removing one or both of the first turn indicator 437 and the second turn indicator 444, altering or removing the path 435 (e.g., increasing the spacing between adjacent “breadcrumbs” along the path 435), altering or removing the visual indicator 454, and so forth. Other techniques for adjusting the difficulty are also contemplated.

In other embodiments, the next task may be may be selected from a plurality of candidate tasks, or may be dynamically generated, based on a stronger correlation of the candidate task with the symptom(s) whose overall score(s) did not meet the threshold for a conclusive assessment.

Performance of the one or more additional tasks may continue until one or more suitable criteria are met, such as exceeding the threshold for the symptom(s), reaching a maximum number of tasks, having less than a minimum amount of change in the overall score per iteration, and so forth.

When the cognitive disorder assessment code 245 determines, at block 355, that no additional tasks are needed (“NO”), flow proceeds to block 365, where the cognitive disorder assessment code 245 reports, based on the performance of the first task (and the performance of the one or more additional tasks when needed), an assessment of whether the user exhibits any of the one or more symptoms. The assessment may be presented in any suitable form, such as reporting the values of the set of predetermined symptoms (or of the ten warning signs), reporting those symptoms having a conclusive assessment, reporting an overall characterization of the one or more symptoms, and so forth. The assessment may further include a recommendation as to whether the user should seek a formal medical assessment. The method 300 ends following completion of block 365.

While the foregoing is directed to embodiments of the present invention, other and further embodiments of the invention may be devised without departing from the basic scope thereof, and the scope thereof is determined by the claims that follow.

1. A method comprising:
prompting a user to perform a first task within a computer-generated virtual environment, the first task being suggestive of whether the user exhibits one or more symptoms of a set of predetermined symptoms indicative of a cognitive disorder;
assessing performance of the first task based on user interaction data generated within the virtual environment and received via one or more input devices, the user interaction data including at least one of navigation behavior, timing information, or response accuracy;
determining, based on the performance of the first task, including evaluating the performance against at least one threshold associated with the one or more symptoms, whether performance of one or more additional tasks is needed to conclusively assess whether the user exhibits any of the one or more symptoms, wherein the one or more additional tasks are selected or generated based on the one or more symptoms not conclusively assessed from the first task; and
reporting, within the virtual environment or via an associated interface, based on the performance of the first task and the performance of the one or more additional tasks when needed, an assessment of whether the user exhibits any of the one or more symptoms.

2. The method of claim 1, wherein the first task includes navigating an avatar to a predefined location within the virtual environment.

3. The method of claim 2, wherein the prompting of the user to perform the first task includes presenting visual guidance to the predefined location.

4. The method of claim 2, wherein the performance of one or more additional tasks is needed, the method further comprising:
prompting the user to perform a second task within the virtual environment, wherein the second task includes navigating the avatar to the predefined location; and
assessing performance of the second task.

5. The method of claim 4, wherein the assessing performance of the second task comprises one of:
determining whether the second task is completed within a time criterion; and
when prompting the user to perform the first task includes presenting visual guidance to the predefined location, determine whether the user completed the second task without the visual guidance.

6. The method of claim 1, wherein the cognitive disorder is Alzheimer's disease, wherein the set of predetermined symptoms indicative of Alzheimer's disease comprises one or more of:
forgetting recently presented information;
decreased concentration;
difficulty completing a repeated task;
confusion about time or place;
difficulty with vision;
misplacing items; and
inability to retrace a path.

7. The method of claim 1, further comprising:
accessing historical interaction information for the user within the virtual environment; and
generating the first task based on the historical interaction information.

8. A system comprising:
one or more computer processors configured to:
prompt a user to perform a first task within a virtual environment, the first task being suggestive of whether the user exhibits one or more symptoms of a set of predetermined symptoms indicative of a cognitive disorder;
assess performance of the first task responsive to user inputs received from one or more input devices;
determine, based on the performance of the first task, whether performance of one or more additional tasks is needed to conclusively assess whether the user exhibits any of the one or more symptoms; and
report, based on the performance of the first task and the performance of the one or more additional tasks when needed, an assessment of whether the user exhibits any of the one or more symptoms.

9. The system of claim 8, wherein the first task includes navigating an avatar to a predefined location within the virtual environment.

10. The system of claim 9, wherein the prompting of the user to perform the first task includes presenting visual guidance to the predefined location.

11. The system of claim 9, wherein the performance of one or more additional tasks is needed, the one or more computer processors further configured to:
prompt the user to perform a second task within the virtual environment, wherein the second task includes navigating the avatar to the predefined location; and
assess performance of the second task.

12. The system of claim 11, wherein the assessing performance of the second task comprises one of:
determining whether the second task is completed within a time criterion; and
when prompting the user to perform the first task includes presenting visual guidance to the predefined location, determine whether the user completed the second task without the visual guidance.

13. The system of claim 8, wherein the cognitive disorder is Alzheimer's disease, wherein the set of predetermined symptoms indicative of Alzheimer's disease comprises one or more of:
forgetting recently presented information;
decreased concentration;
difficulty completing a repeated task;
confusion about time or place;
difficulty with vision;
misplacing items; and
inability to retrace a path.

14. The system of claim 8, the one or more computer processors further configured to:
access historical interaction information for the user within the virtual environment; and
generate the first task based on the historical interaction information.

15. A computer program product comprising:
one or more computer-readable storage media; and
program instructions stored on the one or more computer-readable storage media to perform operations comprising:
prompting a user to perform a first task within a virtual environment, the first task being suggestive of whether the user exhibits one or more symptoms of a set of predetermined symptoms indicative of a cognitive disorder;
assessing performance of the first task responsive to user inputs received from one or more input devices;
determining, based on the performance of the first task, whether performance of one or more additional tasks is needed to conclusively assess whether the user exhibits any of the one or more symptoms; and
reporting, based on the performance of the first task and the performance of the one or more additional tasks when needed, an assessment of whether the user exhibits any of the one or more symptoms.

16. (canceled)

17. (canceled)

18. (canceled)

19. (canceled)

20. (canceled)

21. The system of claim 8, wherein the one or more computer processors are further configured to determine the performance of the first task by calculating a symptom-specific score based on a weighted combination of multiple types of user interaction data captured during execution of the first task.

22. The system of claim 8, wherein the one or more computer processors are further configured to determine whether performance of one or more additional tasks is needed by comparing performance of the first task to a plurality of thresholds respectively associated with different symptoms.

23. The system of claim 8, wherein the one or more computer processors are further configured to dynamically generate the one or more additional tasks based on historical interaction data of the user and correlations between candidate tasks and respective symptoms.

24. The system of claim 8, wherein the virtual environment comprises a three-dimensional environment rendered for display to the user, and wherein the one or more computer processors are further configured to track spatial movement of a user-controlled avatar through the virtual environment and evaluate performance based on deviations from a prescribed navigation path combined with timing and response accuracy.

25. The system of claim 8, wherein the one or more computer processors are further configured to:
(i) select or generate a sequence of tasks having progressively varied levels of assistance within the virtual environment;
(ii) modify at least one parameter of the virtual environment including visual guidance, spatial cues, or timing constraints; and
(iii) iteratively reassess performance after each task using updated interaction data, wherein the determination of whether the user exhibits the one or more symptoms is based on cumulative performance across the sequence of tasks.
